(19) United States
(12) Patent Application Publication
(10) Pub. No.: US 2006/0099277 A1
(43) Pub. Date: May 11, 2006

(54) Protein and fruit juice product

(76) Inventors: Fred F. Jewett JR., Rowley, MA (US); Jayne M. Minigell, Andover, MA (US); Robert C. Jones, Belmont, MA (US); Steven P. Bullens, Worcester, MA (US); Joe C. W. Ho, Tuen Mun, New Territories (CN)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/251,073

(22) Filed: Oct. 14, 2005

Related U.S. Application Data
(60) Provisional application No. 60/619,516, filed on Oct. 15, 2004.

(51) Int. Cl. A61K 36/82

(52) U.S. Cl. 424/729

(57) ABSTRACT

The invention is a product that produces a feeling of satiety, suppresses appetite and promotes a higher metabolic rate, while providing nutrients for a balanced meal in a fruit flavored meal substitute. The product for weight management comprises fruit juice and protein, and further comprises green tea extract comprising epigallocatechin gallate. The product can be made to further comprise caffeine at a level over and above that provided by the green tea extract, and added calcium. Other aspects of the invention include methods to provide a human with a product that aids in the feeling of satiety and contributes to weight management, comprising administering to a human in need thereof the food product described.

### RELATED APPLICATION

This application claims the benefit of U.S. Provisional Application No. 60/619,516, filed Oct. 15, 2004. The entire teachings of the above application are incorporated herein by reference.

### BACKGROUND OF THE INVENTION

The prevalence of obesity in adults, children and adolescents has increased rapidly over the past 30 years in the United States and globally and continues to rise. Overweight and obesity are associated with increasing the risk of developing many chronic diseases of aging seen in the U.S. (Must, A. et al., JAMA, 282: 1523-9 (1999)). Such co-morbidities include type 2 diabetes mellitus, hypertension, coronary heart diseases and dyslipidemia, gallstones and cholecystectomy, osteoarthritis, cancer (of the breast, colon, endometrium, prostate, and gallbladder), and sleep apnea. It is estimated that there are around 325,000 deaths annually that are attributable to obesity. The key to reducing the severity of the diseases is to lose weight effectively or to prevent weight gain. Although about 30 to 40% claim to be trying to lose weight or maintain lost weight, current therapies appear not to be working. Besides dietary manipulation, pharmacological management and in extreme cases, surgery, are sanctioned adjunctive therapies to treat overweight and obese patients (Expert Panel, National Institute of Health, Heart, Lung, and Blood Institute, 1-42 (June 1998); Bray, G. A., Contemporary Diagnosis and Management of Obesity, 246-273 (1998)). Drugs have side effects, and surgery, although effective, is a drastic measure and reserved for morbidly obese.

New options are needed to help overweight people lose weight and maintain the weight loss. There is need for a ready-to-drink, good tasting product for individuals choosing to lose and/or maintain weight.

### SUMMARY OF THE INVENTION

The invention is a product for weight management comprising fruit juice, protein, and further comprising green tea extract which contains epigallocatechin gallate (EGCG). The product can be made to further comprise caffeine at a level over and above that provided by the green tea extract.

The invention is also a product for weight management comprising fruit juice, protein, added calcium, and further comprising green tea extract which contains epigallocatechin gallate (EGCG). The product can be made to further comprise caffeine at a level above that provided by the green tea extract.

One aspect of the invention is a product for weight management, which comprises fruit juice, protein derived from soybeans or from another source or from a combination of protein sources, fiber, calcium, fat, caffeine, and further comprises green tea extract which includes epigallocatechin gallate. The green tea extract also comprises catechin polyphenols and caffeine. The product can also contain added caffeine to augment the caffeine found in the green tea extract.

A particular aspect of the invention is a product for weight management, which comprises from about 20% to about 60% fruit juice by weight, from about 6 grams to about 14 grams protein per serving of product, one or more sources of fiber providing from about 3 grams to about 10 grams fiber per serving, one or more sources of calcium providing from about 300 mg to about 1,000 mg dietary calcium per serving, about 4 grams or less of fat, and further comprises an amount of green tea extract per serving which includes from about 94 mg to about 176 mg epigallocatechin gallate. The green tea extract also comprises catechin polyphenols. The green tea extract provides some amount of caffeine for the product, but additional caffeine can be added, if necessary, to a level to provide from about 40 mg to about 110 mg caffeine per serving.

The product can use for its source of protein, soymilk, soymilk powder, soy protein concentrate, soy protein isolate, protein from dairy whey, casein, whey protein concentrate, cereals or eggs, or a combination of two or more of any the foregoing in any proportion.

The product can further comprise one or more sweeteners, which can be natural sugars or artificial sweeteners.

The product can contain fruit juice chosen for low glycemic index.

In the product, the source of calcium can be, for example, calcium citrate, calcium phosphate, calcium carbonate, calcium malate, calcium fumarate, calcium lactate or calcium acetate, or a combination of the foregoing.

The source of fiber can be, for example, soy, inulin, oat, barley, wheat, guar, pectin, xanthan carrageenan, konjac, gum arabic or fruit pulp, or a combination of the foregoing.

The product comprises one or more fats. The fat can be provided from, for instance, soybean oil, coconut oil, canola oil, cow's milk, goat's milk, cocoa, sunflower seed oil, safflower oil, olive oil, flaxseed oil, borage oil or hemp oil, or a combination of any of the foregoing.

The product for weight management can be provided in a beverage of serving size of from about 8 fluid ounces to about 12 fluid ounces, or in a pudding or frozen dessert of serving size of from about 5 ounces to about 10 ounces.

In one embodiment of the invention, the product is provided in a beverage with a viscosity of from about 150 to about 300 centipoise.

In particular embodiments of the invention, the product contains from about 40 mg to about 110 mg, from about 64 mg to about 96 mg, or from about 60 to 100 mg caffeine per serving.

In another particular embodiment, the product contains from about 108 mg to about 162 mg epigallocatechin gallate per serving.

The product can be formulated to contain a source of calcium providing from about 300 mg to about 1,000 mg dietary calcium per serving, from about 300 mg to about 500 mg dietary calcium per serving, from about 500 mg to about 700 mg dietary calcium per serving, or from about 700 mg to about 900 mg dietary calcium per serving, and preferably provides about 500 mg dietary calcium per serving.

In a particular formulation, the product provides from about 150 to about 300 calories per serving.

The product can be fortified with additional sources of a vitamin or mineral selected from the group consisting of: vitamin A, vitamin D, vitamin K, riboflavin, vitamin B₆, vitamin B₁₂, pantothenic acid, iodine, zinc, copper, chromium, vitamin C, iron, vitamin E, thiamin, niacin, folate, biotin, phosphorus, selenium, manganese and molybdenum and magnesium, or a combination of any of the foregoing.

Another aspect of the invention is a method of providing a human with a product that aids in the feeling of satiety, comprising administering to a human in need thereof a product which comprises from about 20% to about 60% fruit juice by weight, from about 6 grams to about 14 grams protein per serving of product, one or more sources of fiber providing from about 3 grams to about 10 grams fiber per serving, one or more sources of calcium providing from about 300 mg to about 1,000 mg dietary calcium per serving, about 4 grams or less of fat, from about 40 mg to about 110 mg caffeine per serving, and further comprises an amount of green tea extract per serving which includes from about 94 mg to about 176 mg epigallocatechin gallate. The green tea extract also comprises catechin polyphenols.

Further, the invention is a method of providing a human with a product that aids in the management of weight gain and promotes weight loss, comprising administering to a human in need thereof the product as described above in any of its variations.

Yet another aspect of the invention is a method of providing a human with a product that aids in the prevention of weight gain, comprising administering to a human in need thereof the product as described above in any of its variations.

The methods of the invention can be carried out by administering the product of the invention in two daily servings, with serving sizes as described herein.

### DETAILED DESCRIPTION OF THE INVENTION

A description of preferred embodiments of the invention follows.

The invention is a product containing protein, fruit juice, fiber, green tea components, including epigallocatechin gallate (EGCG) and caffeine, the green tea components at least in part being provided in a green tea extract. The product is supplemented with calcium, and will contain a low amount of fat. The product can contain additional sweeteners, vitamin, mineral, micronutrient and nutraceutical supplements, and other components to contribute to taste, texture and appearance.

The product can be used as a meal replacement for weight management, producing the effect of appetite suppression, increased satiety, and increased resting metabolic rate. The product can be supplied as a ready to eat product in the form of a beverage, pudding or frozen dessert product, for example, or in a semi-solid or solid form as a food bar.

The protein of the product is provided from soymilk, soymilk powder, soy protein concentrate, dairy whey, casein, soy, milk, egg, or cereal proteins or from combinations thereof. Proteins from a high biological value source are preferred as they promote protein synthesis (Crim, M. C. et al., Modern Nutrition in Health and Disease, ch. 2: 3-36 (1994)). A smooth, thin, refreshing mouth feel is a desirable characteristic for the food products of the instant invention. Protein characteristics play a major role in the mouth feel of a food product. Low viscosity soy protein is one source of protein. Soy protein viscosity is related to degree of hydrolysis. A preferred degree of hydolysis (DH) is below about 15%, more preferably from about 1 to about 6%.

Soymilk has been prepared for hundreds of years by a traditional water extraction method which involves soaking the soybeans in water for several hours, draining, grinding with additional water, filtering to remove the insoluble residue and cooking the filtrate for about 30 minutes. Methods to prepare soymilk are known in the art. A typical method to prepare soymilk comprises (a) washing soybeans; (b) soaking the soybeans; (c) rinsing the beans to dehull them; (d) chopping the beans and adding water thereto to form a slurry thereof; (e) cooking the beans; (f) cooling the beans; and (g) extracting the milk.

For improving the flavor of soymilk, several methods were developed in the United States during the 1970s, wherein soybeans are ground while applying heat, thereby deactivating lipoxygenases and preventing the formation of soybean odor components. These methods are called the “Cornell method,” “USDA method” and “Illinois method,” respectively. Several methods are compared in Journal of Food Science, 43:349-353 (1978). Any of the known methods in the art are acceptable for the preparation of soymilk. “Soymilk powder” is generally the product obtained by removal of water from liquid soymilk, or by the blending of edible-quality soy protein and soybean oil powders.

The fruit juice component can be derived from, for example, whole fruit, juice concentrates, fruit puree or blends thereof, including apple, apricot, banana, blackberry, blueberry, boysenberry, melon, cherry, cranapple, cranberry, current, elderberry, grape, grapefruit, honeydew, kiwi, lemon, lime, mango, nectarine, orange, papaya, passion fruit, peach, pineapple, plum, pomegranate, prune, raspberry, strawberry, tangerine, tomato, and watermelon. Fruit juices in the lower range of glycemic index values among fruit juices are preferred.

The “glycemic index” is a system of predicting subsequent rises in blood glucose after ingestion of carbohydrate-containing foods (Anderson, J. S. et al., Modern Nutrition in Health and Disease, ch. 70: 1259-86 (1994); Wolever, T. M. S. et al., Am. J. Clin. Nutr., 54: 846-54 (1991); Wolever, T. M. S. et al., Diab. Care, 12: 126-32 (1990)). The glycemic index characterizes the rate of carbohydrate absorption after a meal. It is defined as the area under the glycemic response curve during a 2-hour period after consumption of 50 g of carbohydrate from a test food divided by the area under the curve of a standard, which is either white bread or glucose. The high glycemic index carbohydrates have the highest peak circulating glucose in a 2 hour period following ingestion of food. Conversely, low glycemic index carbohydrates cause a lower peak glucose and a smaller area under the curve.

Consumption of low glycemic index carbohydrates can contribute to weight loss through energy intake regulation (Ludwig, D. S., J. Nutr., 130:280S-283S (2000); Roberts, S. R., Nutr. Rev., 58:163-169 (2000)). Increased satiety, a delay in return to a state of hunger and a decrease in food intake at a subsequent meal occurs with ingestion of low glycemic index carbohydrates. Low glycemic index values are defined by Brand-Miller et al. as 0-55. See Brand-Miller, J. et al., The New Glucose Revolution, Marlowe & Company, New York, 2003, especially pages 32-33.

Recently, green tea has been shown to induce weight loss (Dulloo, A. G. et al., Am. J. Clin. Nutr., 70: 1040-5 (1999); Juhel, C. et al., J. Nutr. Biochem., 11: 45-51 (2000)). The mechanism for increasing energy expenditure by green tea has been postulated to be its flavonoid, and more specifically its polyphenolic content (Dulloo A. G. et al., Am. J. Clin. Nutr., 70: 1040-5 (1999)). One class of these compounds, the catechins, have been shown to inhibit catechol O-methyltransferase (COMT), an enzyme that degrades norepinephrine. This inhibition allows norepinephrine to exert a prolonged influence on thermogenesis and fat metabolism. Both of these metabolic processes are controlled by the sympathetic nervous system via norepinephrine. The delay in degrading norepinephrine allows for it to remain in the sympathetic synaptic cleft longer and exert its effect. Caffeine also has an effect on norepinephrine by inhibiting phosphodiesterases and prolonging the life of cAMP in the cell. These actions coupled with the sustained effect of norepinephrine caused by epigallocatechin gallate (EGCG) affect thermogenesis (Dulloo, A. G. et al., Am. J. Clin. Nutr., 49: 44-5 (1989); Dulloo, A. G. et al., Am. J. Clin. Nutr., 70: 1040-5 (1999)). Although there are numerous catechins in green tea, EGCG is probably the most influential. It cannot be obtained in appreciable amounts from any other food source. In addition, green tea extract has been shown in vitro to inhibit gastric and pancreatic lipases by 37% (Juhel, C. et al., J. Nutr. Biochem., 11: 45-51 (2000)). One weight loss product on the market today (orlistat) also induces weight loss by the same mechanism and it has been proven to be efficacious (Hill, J. O. et al., Am. J. Clin. Nutr., 69: 1108-16 (1999)).

The amount of green tea extract incorporated into the product should be that which provides from about 94 to about 176 mg epigallocatechin gallate (EGCG), from about 108 to about 162 mg EGCG, or from about 121 to about 149 mg EGCG per serving, but preferably, about 135 mg EGCG, per serving. It is preferable for two servings per day to be consumed, with a daily intake of about 270 mg EGCG. Green tea extract is also a source of caffeine. Additional caffeine can be incorporated into the product in combination with green tea extract, particularly if the green tea extract does not provide adequate levels of caffeine. The amount of caffeine per serving should be within the range of 40 mg to 110 mg, and can be from about 56 to about 104 mg, from about 64 to about 96 mg, or from about 72 to about 88 mg, but preferably, about 75 or 80 mg.

The fat of the product can be, for example, a non-atherogenic oil, preferably a vegetable oil selected from the group consisting of: canola, olive, soy, safflower, sunflower, corn and combinations thereof.

Dietary fiber is understood to be all of the components of a food that are not broken down by enzymes in the human digestive tract to produce small molecular compounds which are then absorbed into the bloodstream. These components are mostly celluloses, hemicelluloses, pectin, gums, mucilages, lignin and lignin material varying in different plants according to type and age. These fibers differ significantly in their chemical composition and physical structure and consequently their physiological function. One type of dietary fiber which has been used in liquid nutritional products is soy polysaccharide. In the present invention, the preferred sources of dietary fiber include soy, inulin (for example, from chicory), cereal fibers such as those from oat, barley or wheat, hydrocolloids such as guar, pectin, xanthan, carrageenan, konjac mannan or gum arabic, or fruit pulp. Konjac mannan has a low glycemic index, increasing satiety in obese and non-obese patients with type 2 diabetes (Doi, K. et al., Progress in Obesity Research, ch. 80: 507-14, (1990)).

The “RDI for vitamins and minerals” refers to the list published in the 1993 Federal Register, Vol. 58. RDIs are a set of dietary references based on the Recommended Dietary Allowances (RDA) for essential vitamins and minerals. The name “RDI” replaces the term, “U.S. RDA” (Recommended Daily Allowances). Recommended Dietary Allowances (RDA) are the set of estimated nutrient allowances established by the National Academy of Sciences used as the basis for setting the U.S. RDAs. It is updated periodically to reflect current scientific knowledge.

Vitamins and minerals can be included in the product of the invention such that a significant proportion of the Reference Daily Intake (RDI) is supplied in a serving. In one embodiment of the invention, a single serving of the product can provide about 50% of the RDI for calcium, about 35% of the RDI for vitamin A, vitamin D, viatmin K, riboflavin, vitamin B₆, vitamin B₁₂, pantothenic acid, iodine, zinc, copper, chromium, vitamin C, iron, vitamin E, thiamin, niacin, folate, biotin, phosphorus, selenium, manganese and molybdenum, and about 15% of the RDI for magnesium.

The product can also contain other ingredients such as one or a combination of other vitamins, minerals, antioxidants, fiber, micronutrients and herb supplements (e.g., ginkgo biloba, ginseng, beta-carotene, inositol) and other nutritional supplements. Selection of one or several of these ingredients is a matter of formulation design, consumer and end-user preference. The amounts of these ingredients added to the products of this invention are readily known to the skilled artisan.

The food product of the present invention can optionally include one or more flavorings and/or natural or artificial color agents to complement the natural juice flavor utilized. If soy protein is used, additional flavoring can help to mask the beany/nutty/bitter flavor elements in soy protein.

The product can be made in forms that are alternatives to the beverage, and which may contain less water than the beverage, such as puddings, products resembling yogurt, ice cream, and other frozen confections, novelties and desserts, and in the form of food bars. The alternative forms are prepared by methods known in the art, and may contain, in addition to the components described for the beverage, agents to thicken the product, usually to a concentration of 2% by weight, or less. Such agents can include, for example, hydrocolloids such as gelatin, or such as the polysaccharides gum arabic, guar gum carboxymethylcelulose, xanthan, carrageenan, agar, alginates, or pectins. Frozen products are cooled in an ice cream freezer.

Product stability and mouth feel can be improved by using one or more stabilizers. Various food stabilizers can be employed in the present invention and include hydrophilic colloidal stabilizers known as gum arabic, pectins, gelatin, and xanthan as well as the anionic polymers derived from cellulose such as carboxymethyl cellulose. These stabilizers are water soluble and tolerate a low pH.

Pectins can be preferably used in the present invention to enhance physical stability and control the viscosity of the food products. The pectin stabilizes soy proteins to yield products without significant sedimentation and phase separation (i.e., physical stability). Pectin also ensures a smooth mouth feel without any “sandiness” and helps to control viscosity by stabilizing free water. Pectins are a class of complex polysaccharides found in the cell walls of higher plants. The number of plant sources that are used for the commercial production of pectins is fairly limited. At present, apple pomace and citrus peels are the main sources of commercially acceptable pectins. They, however, produce slightly different pectins, which make one or the other more suitable for specific applications. Other sources of pectin include sugar beets and the seed heads of sunflowers.

An important property of pectins is their ability to form gels in the presence of Ca⁺⁺ ions or sugars and acid. Depending on the degree of methoxylation (DM), pectins are classified into: 1) low methoxy (LM) pectin with a DM of 25-50% and 2) high methoxy (HM) pectin of 50-80% DM. LM and HM pectins form gels in the presence of calcium ions and acid, respectively. The mechanism of gel formation is different in both HM and LM pectins.

The pH of the beverages and other products of this invention should range from about 3.0 to about 8.0. This pH range can be obtained by the addition of food grade acids such as hydrochloric acid, malic acid, citric acid, acetic acid, tartaric acid, lactic acid, lemon juice, lemon puree, lime puree, cranberry juice, orange juice, pineapple juice, apple juice, grapefruit juice, phosphoric acid or mixtures thereof. Any food grade acid known in the art may be utilized.

Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product.

Preservatives may also be added to the product to extend product shelf life. If desired, preservatives such as potassium sorbate, sodium sorbate, potassium benzoate, sodium benzoate or calcium disodium EDTA can be used.

The product can contain artificial sweeteners, e.g., saccharides, cyclamates, aspartamine, aspartame, acesulfame K, and sorbitol. Such artificial sweeteners can be substituted, all or in part, for natural sugars such as sucrose, fructose, glucose and lactose, to minimize caloric content of the product, and could be more suitable for individuals with type II diabetes who are prone to hyperglycemia. If a natural sweetener is to be used, fructose is a preferred carbohydrate. It is sweeter than ordinary table sugar (sucrose) derived from beet or cane sugars, and has a low glycemic index (GI=32).

The product can be produced by a procedure that follows the general steps described below. A high pressure homogenization step is preferred.

Heated water and the source(s) of protein are added to a mixer. A separate pectin solution is prepared in hot water and cooled to room temperature. Pectin solution and fruit juice concentrate are added to the protein in the mixer. Other ingredients such as flavor agents, fiber, vitamins, minerals, green tea extract and caffeine are added to the mixer. The pH is adjusted to the optimal value, preferably using fruit juice acids. A color agent is added. The batch is heated to a high temperature (e.g., 190-200 ° F.) for a short period of time (e.g., 30 seconds), then put through a high pressure homogenizer once. The batch can then be cooled and packaged.

Obesity is a heterogeneous group of conditions with multiple causes (Kopelman P. G., Nature, 404: 635-43 (2000)). Body weight is determined by an interaction of genetics, the environment, and energy balance (i.e., the relationship between energy intake and energy expenditure). Energy expenditure has several components. The major one, basal metabolism, accounts for up to two-thirds of the daily total energy needs (Bray, G. A., Contemporary Diagnosis and Management of Obesity, 35-67 (1998)). This includes energy to maintain body temperature, contracting smooth muscles of the heart and gastrointestinal tract, and mobilization of substances like food and oxygen across cell membranes. Another one-tenth of the energy expenditure is dissipated through the thermic effect of food (energy cost of digestion, absorption, and metabolism of food), which is reduced in obesity. Lastly, exercise (physical activity) contributes to energy expenditure, which represents about 20% to 50% of the total (Kopelman, P. G., Nature, 404: 635-43 (2000)).

Providing the product of the invention to a human can counteract the tendency to gain weight, by increasing a feeling of satiety while providing a large proportion of the daily nutritional needs in a good-tasting, satisfying meal substitute of pleasing consistency. The active ingredients in the product work to increase the body's rate of energy expenditure. Caffeine, EGCG and the other active components of green tea increase the rate at which the body burns calories at rest. The carbohydrate component of the product is specifically chosen to manage blood glucose levels and increase satiety, delaying the return of hunger. Protein and fat create a product with balanced nutrients. Protein has long been known as a satiating macronutrient. Recent studies have shown that a high intake of calcium, and possibly calcium in combination with protein, as can be found in dairy products, for example, may contribute to metabolic responses that result in reduced food intake (Ping-Delfos, W C et al., Asia Pac. J. Clin. Nutr. 2004;13(Suppl):S82). A satisfying amount of protein, in combination with calcium, is provided in the product of the invention. This combination produces appetite suppression.

The products of the invention are intended to be orally administered daily as a meal replacement for weight management. Based on the serving size of 8-12 fluid ounces of beverage or an amount of pudding or frozen dessert product equivalent to 5-10 fluid ounces of the product in beverage form, the recommended dosage is once or twice daily to produce and maintain a feeling of satiety. As used herein, satiety refers to the sensation of fullness between one meal and the next.

### EXAMPLE

Below are some parameters to describe a mango-flavored beverage that is one embodiment of the invention.

Mango Smoothie

| | |
|---|---|
| total calories: | 200 |
| 40% juice | |
| Target pH: | 4.4 |
| serving size (g): | 306.68 |
| serving size (fl oz): | 10 |
| protein/serving (g): | 10 |
| finished product specific gravity: | 1.037 |
| soy protein/serving target %: | 10 g/3.5% |
| juice concentrate type: | mango concentrate and apple juice |

Below are listed the steps in an example of a process to produce a mango smoothie beverage of the invention.
1. Add 85% of formula water (160° F.) to batch mixer; save 10% water for rinsing.
2. Mix Isolated Soy Protein (ISP) and soy milk powder with water in batch mixer.
3. Mix ISP and soy powder for 5 minutes.
4. Make up a 4% pectin solution separately using hot water (170° F.; use 5% of batch water). Blend pectin and water at low speed for 1 minute, and cool to ambient temperature.
5. Add the pectin solution, fruit concentrates and the chicory nectar to the mixer (may have to increase mixing speed at this point because the fruit juice will thicken the batch).
6. Add natural flavors, fibers, vitamins and nutraceuticals (i.e., green tea extract, caffeine; etc.) to mixer (use remaining water for rinsing).
7. Mix for 2 minutes or until well blended.
8. Adjust to target pH with sufficient malic (or other fruit) acid(s). Initial pH was 5.6; adjusted to 4.41.
9. Add natural colors to obtain target mango color.
10. Heat process batch to 190-200° F., 30 sec hold time.
11. Homogenize at up to 9000 psi, 1 pass.
12. Cool to <40° F., and pack.

Major ingredients in the mango smoothie, listed in approximate order of weight percent of the beverage, from greatest to least:
water, fruit juice from concentrate, (apple, mango), chicory nectar, dried soymilk, green tea, isolated soy protein, inulin (chicory fiber), natural flavors, oat fiber, malic acid, fruit and vegetable juice for color, pectin, sodium choride.

Vitamins and minerals to be added in step 6 of the process above:
dicalcium phosphate, tricalcium citrate, magnesium citrate, sodium ascorbate, vitamin E acetate, niacinamide, ferric pyrophosphate, zinc sulfate, vitamin A palmitate, D-calcium pantothenate, vitamin B₆, manganese sulfate, copper gluconate, riboflavin (vitamin B₂), thiamin, folic acid, biotin, postassium iodide, chromium picolinate, vitamin D₂, vitamin K, sodium molybdenate, sodium selenite, vitamin B₁₂.

The approximate weights per serving and Daily Reference Values (DRVs) for some classes of nutrients and are listed below as an illustration of the composition of a mango smoothie.

| | % Daily Value |
|---|---|
| Total Fat 1.0 g | 1% |
| Saturated Fat 0 g | 0% |
| Trans Fat 0 g | |
| Polyunsaturated Fat 0 g | |
| Monounsaturated Fat 0 g | |
| Cholesterol 0 mg | |
| Sodium 100 mg | 4% |
| Potassium 150 mg | 4% |
| Total Carbohydrate 39 g | 15% |
| Dietary Fiber 8 g | 32% |
| Sugars 31 g | |
| Protein 10 g | |

It is estimated that 10 fluid ounces provide 200 calories, with 10 of those calories from fat.

While this invention has been particularly shown and described with references to preferred embodiments thereof, it will be understood by those skilled in the art that various changes in form and details may be made therein without departing from the scope of the invention encompassed by the appended claims.

What is claimed is:

1. A product for weight management, comprising from about 20% to about 60% fruit juice by weight, from about 6 grams to about 14 grams of protein per serving of product, and further comprising an amount of green tea extract per serving comprising from about 94 mg to about 176 mg epigallocatechin gallate, said green tea extract further comprising catechin polyphenols and caffeine.

2. The product of claim 1 further comprising one or more sources of calcium providing from about 300 mg to about 1,000 mg dietary calcium per serving.

3. The product of claim 2 further comprising one or more sources of fiber providing from about 3 grams to about 10 grams fiber per serving, about 4 grams or less of fat, and from about 40 mg to about 110 mg caffeine per serving.

4. A product for weight management, comprising from about 20% to about 60% fruit juice by weight, from about 6 grams to about 14 grams protein per serving of product, one or more sources of fiber providing from about 3 grams to about 10 grams fiber per serving, one or more sources of calcium providing from about 300 mg to about 1,000 mg dietary calcium per serving, about 4 grams or less of fat, from about 40 mg to about 110 mg caffeine per serving, and further comprising an amount of green tea extract per serving comprising from about 94 mg to about 176 mg epigallocatechin gallate, said green tea extract further comprising catechin polyphenols.

5. The product of claim 4 wherein the source of the protein is soymilk, soymilk powder, soy protein concentrate, or soy protein isolate, or a combination of two or more of the foregoing.

6. The product of claim 4 further comprising protein from dairy whey, casein, whey protein concentrate or egg, or a combination of two or more of the foregoing.

7. The product of claim 4 which further comprises one or more sweeteners.

8. The product of claim 4 wherein the fruit juice has a low glycemic index.

9. The product of claim 4 wherein the source of calcium is calcium citrate, calcium phosphate, calcium carbonate, calcium malate, calcium fumarate, calcium lactate or calcium acetate, or a combination of the foregoing.

10. The product of claim 4 wherein the source of fiber is soy, inulin, oat, barley, wheat, guar, pectin, xanthan carrageenan, konjac, gum arabic or fruit pulp, or a combination of the foregoing.

11. The product of claim 4 wherein the product comprises fat from soybean oil, coconut oil, canola oil, cow's milk, goat's milk, cocoa, sunflower seed oil, safflower oil, olive oil, flaxseed oil, borage oil or hemp oil, or a combination of the foregoing.

12. The product of claim 4 wherein the product is provided in a beverage of serving size of from about 8 fluid ounces to about 12 fluid ounces.

13. The product of claim 4 wherein the product is provided in a pudding or frozen dessert of serving size of from about 5 ounces to about 10 ounces.

14. The product of claim 4 wherein the product is provided in a beverage with a viscosity of from about 150 to about 300 centipoise.

15. The product of claim 4 which comprises from about 64 mg to about 96 mg caffeine per serving.

16. The product of claim 4 which comprises from about 108 mg to about 162 mg epigallocatechin gallate per serving.

17. The product of claim 4 which comprises a source of calcium providing from about 400 mg to about 600 mg dietary calcium per serving.

18. The product of claim 4 which provides from about 150 to about 300 calories per serving.

19. The product of claim 4 which further comprises one or more additional sources of a vitamin or mineral selected from the group consisting of: vitamin A, vitamin D, vitamin K, riboflavin, vitamin B₆, vitamin B₁₂, pantothenic acid, iodine, zinc, copper, chromium, vitamin C, iron, vitamin E, thiamin, niacin, folate, biotin, phosphorus, selenium, manganese and molybdenum and magnesium, or a combination of any of the foregoing.

20. A method of providing a human with a product that aids in the feeling of satiety, comprising administering to a human in need thereof the product of claim 1.

21. A method of providing a human with a product that aids in the management of weight gain and promotes weight loss, comprising administering to a human in need thereof the product of claim 1.

22. A method of providing a human with a product that aids in the prevention of weight gain, comprising administering to a human in need thereof the product of claim 1.

23. The method of claim 20, wherein the product is provided in two daily servings.
